(19) United States
(12) Patent Application Publication
(10) Pub. No.: US 2022/0117264 A1
(43) Pub. Date: Apr. 21, 2022

(54) FERMENTATION BROTHS AND THEIR USE

(71) Applicant: EVONIK OPERATIONS GMBH, Essen (DE)

(72) Inventors: Christos GIATSIS, Münster (DE); Stefan PELZER, Gütersloh (DE); Lorena STANNEK-GÖBEL, Bielefeld (DE); Lukas FALKE, Bielefeld (DE); Frank ERHARDT, Bielefeld (DE)

(73) Assignee: EVONIK OPERATIONS GMBH, Essen (DE)

(21) Appl. No.: 17/421,917

(22) PCT Filed: Jan. 8, 2020
(86) PCT No.: PCT/EP2020/050267

§ 371 Date: Jul. 9, 2021

(30) Foreign Application Priority Data
Jan. 10, 2019 (EP) 19151165.8

(51) Int. Cl. A23K 10/18; A23K 30/20; C12N 1/20

(52) U.S. Cl. CPC A23K 10/18; C12R 2001/07; C12N 1/20; A23K 30/20

(57) ABSTRACT

Summary

According to the invention it was found out that fermentation broths of probiotic microorganisms show beneficial characteristics which make them suitable as feed additives as well as means for improving the properties of other feed additives.

According to the invention it was found out that fermentation broths of probiotic microorganisms show beneficial characteristics which make them suitable as feed additives as well as means for improving the properties of other feed additives.

Probiotic microorganisms are obtained by cultivating a sample of the probiotic microorganisms in a fermentation medium and subsequently separating the probiotic microorganisms from the fermentation broth such obtained.

The remaining fermentation broth is normally discarded. But alternative uses of the fermentation broth have also been disclosed in the literature. For example, U.S. Pat. No. 6,060,051 discloses the use of the remaining fermentation broth for isolating a metabolite which is useful against fungal and bacterial plant diseases and in particular has activity against corn rootworm.

Surprisingly, according to the invention, it was found out that drying of the residual fermentation broth seems not to have a detrimental effect on the activity of the active substances as contained in the residual fermentation broth, in particular on the enzymatic and antimicrobial activities.

Further, drying of the fermentation broth leads to a product which can easily be handled and thus applied as feed additive and mixed with further feed additives.

In particular it turned out that by mixing different dried fermentation broths and/or by mixing a dried fermentation broth with a probiotic microorganism, feed products with superior characteristics can be obtained, in particular with respect to proteolytic activity, antimicrobial activity against pathogens and prebiotic activity with respect to beneficial bacteria.

Thus, a first subject matter of the invention is a method of producing a dried fermentation broth, comprising the following steps:
a) Cultivating microorganisms in a fermentation medium to obtain a fermentation broth containing the microorganisms;
b) Separating at least 20% of the microorganisms from the fermentation broth;
c) Drying the fermentation broth such obtained to obtain a dried fermentation broth.

In the separation step, at least 20% of the microorganisms are removed from the fermentation broth, preferably at least 50, 60, 70, 80, 90 or 95%, more preferably at least 98, 99 or 99.5%. In a very preferred embodiment of the invention, the fermentation broth is free or almost free of any microorganisms.

Separation of the microorganisms from the fermentation broth may be carried out in particular by centrifugation, flotation, filtration, particularly ultrafiltration or microfiltration and/or decanting. Drying of the fermentation broth is preferably carried out by freeze-drying, spray drying, vacuum drying, tray drying, drum drying, fluidized bed drying or spray granulation of the fermentation broth.

Freeze-drying of the fermentation broth can be carried out by first freezing the broth by using liquid nitrogen or dry ice or refrigerating at −20° C. and then drying it under high vacuum (Ananta et al., 2004, Microbial Ecology in Health and Disease, 16(2-3): 113-124). Freeze-drying may also involve evaporative cooling of the fermentation broth (Bond, 2007, pp. 99-107, in Methods in Molecular Biology No. 368. Humana Press, New York, USA).

For spray drying the fermentation broth, fine droplets of the broth, atomized by spraying through a heated nozzle, are sprayed into a drying chamber against hot air. The content of the fermentation broth is collected at the bottom of the chamber (Masters, 1972, Spray drying. Leonard Hill Books, London, UK).

Spray granulation is a preferred process where the fermentation broth is directly converted into free-flowing granulate particles of appropriate particle size.

In a particular embodiment of the invention, either after cultivation of the microorganisms and before separation of the microorganisms and/or after separation of the microorganisms, a concentration step may be carried out to increase the total dry matter of the fermentation broth. Concentration of the fermentation broth may be carried out in particular by solvent evaporation. Solvent evaporation is preferably carried out, if applied, using a rotary evaporator, a thin film evaporator or a falling-film evaporator in a single stage or multistage process.

After removal of the microorganisms from the fermentation broth, the fermentation broth preferably has a solids content (total dry matter) of from 1 to 10% by weight, in particular of from 1 to 6% by weight, and/or preferably contains microorganisms at a concentration of not more than 1×10¹⁰ (cfu) per ml, more preferably at a concentration of not more than 1×10⁹ (cfu) per ml, in particular at a concentration of 1×10⁴ to 1×10⁹ (cfu) per ml or at a concentration of 1×10⁴ to 1×10⁸ (cfu)per ml.

After removal of the microorganisms and before starting the drying of the fermentation broth, specific substances may be added, in particular to preserve the enzymes. These substances may be selected in particular from anti-caking agents, anti-oxidation agents, bulking agents, and/or protectants. Examples of useful substances include polysaccharides (in particular starches, celluloses, methylcelluloses, maltodextrins, gums, dextrans, chitosan and/or inulins), polyethylene glycol, amino acids (in particular proline, glycine and/or glutamic acid) protein sources (in particular peptones, skim-milk powder and/or sweet-whey powder), peptides, sugars (in particular lactose, xylose, fructose, trehalose, sucrose and/or dextrose), polyols (in particular mannitol, glycerol and/or sorbitol), yeast extract, malt extract, soybean flour, lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate).

Preferably at least one of these substances and/or a combination of these substances is added, if used, in an amount, so that they are contained in the supplemented fermentation broth suspension in an amount of between one tenth to twofold, preferably between one fifth to equal, with respect to the total dry matter as contained in the fermentation broth before addition of the substance(s).

The resulting dry products may be further processed, such as by milling or granulation, to achieve a specific particle size or physical format.

Further the microorganisms, which are used to produce the fermentation broth, are preferably probiotic microorganisms, in particular probiotic bacteria, and preferably selected from Bacillus, in particular B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, Enterococcus, in particular E. faecium and E. faecalis, Geobacillus, in particular G. stearothermophilus, Clostridium, in particular C. butyricum, and Streptococcus, in particular S. faecalis, S. faecium, S. gallolyticus, S. salivarius subsp. thermophilus and S. bovis, Lactobacillus, in particular L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius and L. sanfranciscensis, Pediococcus, in particular P. acidilactici, P. dextrinicus and P. pentosaceus, Streptococcus, in particular S. lactis and S. thermophiles, Bifidibacterium, in particular S. adolescentis, B. animalis, B. bifidum, B. breve and B. longum.

In a very preferred embodiment of the invention the probiotic microorganisms are of the genus Bacillus, in particular selected from the following strains and combinations thereof: B. subtilis DSM 32315, B. subtilis DSM 32540, B. subtilis DSM 32592, B. licheniformis DSM 32314, B. pumilus DSM 32539, B. amyloliquefaciens CECT 5940.

A further subject matter of the invention are therefore also dried fermentation broths as obtainable by a method according to the invention.

The dried fermentation broths of the invention preferably contain microorganisms in an amount of not more than 1×10¹¹ (cfu) per g of dried fermentation broth, more preferably in an amount of not more than 1×10¹⁰ (cfu) per g, in particular in an amount of 1×10⁴ to 1×10¹¹ (cfu) per g or in an amount of 1×10⁴ to 1×10¹⁰ (cfu) per g or in an amount of 1×10⁴ to 1×10⁹ (cfu) per g of dried fermentation broth.

A further subject of the invention is therefore also a dried fermentation broth containing microorganisms in an amount of not more than 1×10¹¹ (cfu) per g of dried fermentation broth, more preferably in an amount of not more than 1×10¹⁰ (cfu) per g, in particular in an amount of 1×10⁴ to 1×10¹¹ (cfu) per g or in an amount of 1×10⁴ to 1×10¹⁰ (cfu) per g or in an amount of 1×10⁴ to 1×10⁹ (cfu) per g of dried fermentation broth, wherein the microorganisms are preferably selected from B. subtilis and B. amyloliquefaciens.

The amount of cells (cfu) in the dried fermentation broths of the invention is thus preferably below 1 wt.-%, in particular below 0.5 or 0.2 wt.-%, more preferably below 0.1 wt.-%, in particular below 0.05 or 0.02 wt.-%. In particular embodiments the amount of cells (cfu) in the dried fermentation broths is even below 0.01 wt.-%, in particular below 0.005, 0.002 or 0.001 wt.-%.

A further subject of the invention is therefore also a dried fermentation broth containing cells (cfu) in an amount of below 1 wt.-%, in particular below 0.5 or 0.2 wt.-%, more preferably below 0.1 wt.-%, in particular below 0.05 or 0.02 wt.-%, wherein in particular embodiments the amount of cells (cfu) in the dried fermentation broth is even below 0.01 wt.-%, in particular below 0.005, 0.002 or 0.001 wt.-%, wherein the microorganisms are preferably selected from B. subtilis and B. amyloliquefaciens. The amount of cells (cfu) in weight percent is preferably calculated basing on the number of cells (cfu) as disclosed by Jeong et al. (1990) in Biotechnology and Bioengineering, Vol. 35, pages 160-184.

A further subject matter of the invention is therefore in particular a dried fermentation broth of a microorganism, form which at least 20%, preferably at least 50, 70 or 90%, of the microorganisms have been removed, containing at least one substance selected from anti-caking agents, anti-oxidation agents, bulking agents, and/or protectants, in particular selected from polysaccharides (in particular starches, celluloses, methylcelluloses, maltodextrins, gums, dextrans, chitosan and/or inulins), polyethylene glycol, amino acids (in particular proline, glycine and/or glutamic acid) protein sources (in particular peptones, skim-milk powder and/or sweet-whey powder), peptides, sugars (in particular lactose, xylose, fructose, trehalose, sucrose and/or dextrose), polyols (in particular mannitol, glycerol and/or sorbitol), yeast extract, malt extract, soybean flour, lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate). The at least one substance which is contained in the dried fermentation broth and/or a mixture of such substances is preferably present in the dried fermentation broth in an amount of at least 0.1 wt.-%, more preferably in an amount of at least 0.5 wt.-% or at least 1 wt.-%, in particular in an amount of 0.1 to 67 or 10 to 67 wt.-%, preferably in an amount of 0.5 to 50 or 10 to 50 wt.-%, more preferably in an amount of 1 to 30 or 10 to 30 wt.-%.

According to the invention, “dried fermentation broth” means a fermentation broth which has a total dry matter content of at least 70 wt.-%, more preferably at least 80 wt.-%, above all more than 90 wt.-%, in particular at least 95 wt.-%.

Further subject matter of the invention are also compositions comprising at least two, preferably at least three, in particular two, three, four or five, different kinds of dried fermentation broths, in particular of the bacteria as mentioned before.

According to the invention it was surprisingly further found out that fermentation broths of Bacillus amyloliquefaciens exhibit unexpected beneficial characteristics like a very high proteolytic activity.

A further aspect of the invention is therefore a fermentation broth of Bacillus amyloliquefaciens. A further subject matter of the invention are therefore also compositions, in particular feed compositions, containing a fermentation broth of B. amyloliquefaciens, wherein the fermentation broth is preferably a fermentation broth of B. amyloliquefaciens CECT 5940.

The fermentation broth of B. amyloliquefaciens is preferably obtained by cultivating probiotic microorganisms of the species B. amyloliquefaciens in a fermentation medium to obtain a fermentation broth containing said probiotic microorganisms and subsequently separating at least 20%, preferably at least 50, 60, 70 or 80%, more preferably at least 90, 95 or 98%, of the microorganisms from the fermentation broth, so that the fermentation of B. amyloliquefaciens, in particular of B. amyloliquefaciens, is preferably one, from which at least 20, preferably at least 50, 60, 70, 80, 90 or 95% of the microorganisms have been removed.

After removal/separation of the microorganisms, the fermentation broth has preferably a solids content (total dry matter) of from 1 to 10% by weight, in particular of from 1 to 6% by weight, and/or preferably contains microorganisms at a concentration of not more than 1×10¹⁰ per ml, more preferably at a concentration of not more than 1×10⁹ per ml, in particular at a concentration of 1×10⁴ to 1×10⁹ per ml or at a concentration of 1×10⁴ to 1×10⁸ per ml.

In a preferred embodiment of the invention the fermentation broth of B. amyloliquefaciens is used in concentrated or dried form, wherein concentration and/or drying are preferably carried out as disclosed in the description before and/or the concentrated or dried fermentation broth has the characteristics as mentioned before in the description.

A particular subject matter of the invention is therefore also a concentrated and/or a dried fermentation broth of B. amyloliquefaciens, in particular of B. amyloliquefaciens CECT 5940.

A further subject of the invention is therefore also a dried fermentation broth containing B. amyloliquefaciens, in particular B. amyloliquefaciens CECT 5940, in an amount of not more than 1×10¹¹ (cfu) per g of dried fermentation broth, more preferably in an amount of not more than 1×10¹⁰ (cfu) per g, in particular in an amount of 1×10⁴ to 1×10¹¹ (cfu) per g or in an amount of 1×10⁴ to 1×10¹⁰ (cfu) per g or in an amount of 1×10⁴ to 1×10⁹ (cfu) per g of dried fermentation broth.

A further subject of the invention is therefore also a dried fermentation broth containing cells (cfu) of B. amyloliquefaciens, in particular of B. amyloliquefaciens CECT 5940, in an amount of below 1 wt.-%, in particular below 0.5 or 0.2 wt.-%, more preferably below 0.1 wt.-%, in particular below 0.05 or 0.02 wt.-%, wherein in particular embodiments the amount of cells (cfu) in the dried fermentation broth is even below 0.01 wt.-%, in particular below 0.005, 0.002 or 0.001 wt.-%.

The fermentation broth of B. amyloliquefaciens according to the invention has preferably a proteolytic activity of at least 500 mU/ml, more preferably of at least 1000 mU/ml, determined with the method as disclosed in the working examples.

Surprisingly, according to the invention, it was further found out that the properties of a feed can be improved, if an animal feed or an additive thereof is treated with a fermentation broth of microorganisms, before the final feed is prepared.

Another subject matter of the invention is therefore a method of improving the properties of a feed or feed additive, wherein an animal feed or a feed additive is treated/incubated with at least one fermentation broth of at least one microorganism, from which preferably at least 20%, preferably at least 50, 70 or 90%, of the microorganisms have been removed, wherein treatment/incubation is preferably carried out to improve the properties of the final feed product.

The microorganism which is used to produce the fermentation broth, is also here preferably selected from probiotic microorganisms, in particular probiotic bacteria, preferably from Bacillus, in particular B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, Enterococcus, in particular E. faecium and E. faecalis, Geobacillus, in particular G. stearothermophilus, Clostridium, in particular C. butyricum, and Streptococcus, in particular S. faecalis, S. faecium, S. gallolyticus, S. salivarius subsp. thermophilus and S. bovis, Lactobacillus, in particular L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius and L. sanfranciscensis, Pediococcus, in particular P. acidilactici, P. dextrinicus and P. pentosaceus, Streptococcus, in particular S. lactis and S. thermophiles, Bifidibacterium, in particular S. adolescentis, B. animalis, B. bifidum, B. breve and B. longum, wherein in a very preferred embodiment of the invention the probiotic microorganisms are of the genus Bacillus, in particular selected from the following strains and combinations thereof: B. subtilis DSM 32315, B. subtilis DSM 32540, B. subtilis DSM 32592, B. licheniformis DSM 32314, B. pumilus DSM 32539, B. amyloliquefaciens CECT 5940.

The fermentation broth is also for this purpose preferably obtained by first cultivating the microorganisms in a suitable fermentation medium and subsequently separating at least 20, preferably at least 50, 70 or 90%, of the microorganisms from the fermentation broth.

After removal/separation of the microorganisms, the fermentation broth has preferably a solids content (total dry matter) of from 1 to 10% by weight, in particular of from 1 to 6% by weight, and/or preferably contains microorganisms at a concentration of not more than 1×10¹⁰ per ml, more preferably at a concentration of not more than 1×10⁹ per ml, in particular at a concentration of 1×10⁴ to 1×10⁹ per ml or at a concentration of 1×10⁴ to 1×10⁸ per ml.

In a preferred embodiment of the invention the fermentation broth is used in concentrated or dried form, wherein concentration and/or drying are preferably carried out as disclosed in the description before and/or the concentrated or dried fermentation broth has the characteristics as mentioned before in the description.

In particular, it was found out that the amount of antinutritional factors (ANFs) can be significantly reduced, if the feed or an additive thereof is treated with the fermentation broth, before the final feed is prepared.

Raw materials of animal feed as corn and especially soybean meal (SBM) contain ANFs. SBM is a main source of dietary protein in poultry and swine, but is becoming more and more important also as a feed additive for aqua feed. ANFs (e.g. protein inhibitors, non-starch polysaccharides, lectins, antigenic proteins) present in the feedstuff interfere in the utilization of feed nutrients and can cause health problems to the animal. Thus, the ANFs reduction or removal before consumption of the feed is important.

Fermentation of soybean meal by lactobacilli and bacilli is known to reduce ANFs and increase the nutritional value of the feed. There are e.g. hints, that fermentation of SBM by specific bacterial strains can lead to a degradation of the antigenic proteins R-conglycinin and glycinin. These ANFs are believed to be responsible for abnormal morphological changes of the intestine and liver as observed in groupers fed with SBM. Besides reduction of ANFs of the raw material, anti-microbial peptides (AMPs) can be produced during fermentation of the raw material which can inhibit pathogenic bacteria of the host.

As surprisingly it was found out according to the invention, that fermentation broths of microorganisms can also be used to improve the properties of feed additives and in particular to reduce and/or eliminate ANFs in an effective way, thus a further subject matter of the invention is a method of improving the properties of an animal feed or of an animal feed additive, wherein the animal feed or animal feed additive is treated with a fermentation broth of at least one microorganism, from which preferably at least 20%, more preferably at least 50, 70 or 90%, of the microorganisms have been removed.

A preferred subject matter of this aspect of the invention is a method of decreasing the amount of antinutritional factors (ANFs), in particular of ß-conglycinine and/or glycinine, in an animal feed or an animal feed additive, wherein the animal feed or animal feed additive is treated with a fermentation broth of at least one microorganism.

A further preferred subject matter of this aspect of the invention is a method of degrading mycotoxins in an animal feed or animal feed additive, wherein the animal feed or animal feed additive is treated with a fermentation broth of at least one microorganism.

Further improvements of the properties of the feed or feed additive which can be established by the incubation of the feed or feed additive with the fermentation broth are a better usability of the proteins as contained in the feed or feed additive by the animal, preservation of the feed or feed additive, in particular by lowering the pH and/or lowering the amount of contaminating microorganisms in the feed or feed additive. Methods to improve such properties are therefore further preferred embodiments of the invention.

The feed additives which are preferably treated with the fermentation broth to improve their properties and/or the properties of the final feed are preferably selected from corn, soy, barley, rice, oats, sorghum, soybean meal, rapeseed meal and cotton meal.

The fermentation broths of microorganisms which are preferably used according to the invention to treat the animal feed or animal feed additive are selected from fermentation broths of probiotic microorganisms, in particular probiotic bacteria, preferably fermentation broths of probiotic microorganisms as already disclosed above in description, i.e. from Bacillus, in particular B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, Enterococcus, in particular E. faecium and E. faecalis, Geobacillus, in particular G. stearothermophilus, Clostridium, in particular C. butyricum, and Streptococcus, in particular S. faecalis, S. faecium, S. gallolyticus, S. salivarius subsp. thermophilus and S. bovis, Lactobacillus, in particular L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius and L. sanfranciscensis, Pediococcus, in particular P. acidilactici, P. dextrinicus and P. pentosaceus, Streptococcus, in particular S. lactis and S. thermophiles, Bifidibacterium, in particular S. adolescentis, B. animalis, B. bifidum, B. breve and B. longum, wherein in a very preferred embodiment of the invention the fermentation broths are from probiotic microorganisms of the genus Bacillus, in particular selected from the following strains and combinations thereof: B. subtilis DSM 32315, B. subtilis DSM 32540, B. subtilis DSM 32592, B. licheniformis DSM 32314, B. pumilus DSM 32539, B. amyloliquefaciens CECT 5940.

For carrying out the pretreatment of the feed or feed additive, the feed or feed additive and the fermentation broth are preferably mixed in a ratio of 1:2 to 20:1, more preferably 1:1 to 10:1. The preferred mixing ratio depends on whether the fermentation broth is used in liquid, concentrated or dried form, as in the concentrated and dried form the active substances are present in higher concentrations. In case of non-concentrated fermentation broths, the preferred mixing ratio of feed additive to fermentation broth is from 1:2 to 2:1 (on w/w basis), whereas for dried fermentation broths the preferred mixing ratio of feed additive to fermentation broth is from 5:1 to 20:1 (on w/w basis).

To allow an efficient improvement of the properties of the feed or feed additive, incubation of feed or feed additive and fermentation broth is preferably carried out for at least one hour, in particular one hour to 100 hours, more preferably for at least 2 hours, in particular 2 hours to 80 hours, above all for at least 4 hours, preferably 4 hours to 50 hours.

The fermentation broths of the invention, in particular the dried fermentation broths, have preferably at least one, more preferably at least two, three, four, five, six, seven, eight, nine or ten, in particular all, of the following characteristics:
a) Protease activity;
b) Cellulase activity;
c) Xylanase activity;
d) Amylase activity;
e) Phytase activity;
f) Catalase activity;
g) Superoxide dismutase activity;
h) Lactonase activity;
i) Activity against antinutritional factors (ANFs), in particular against ß-conglycinine and/or glycinine;
j) Activity against mycotoxins;
k) Activity against pathogenic microorganisms, in particular against C. perfringens and/or S. suis;
l) Quorum quenching activity;
m) Prebiotic activity with respect to beneficial microorganisms.

In a preferred embodiment of the invention, the fermentation broths of the invention have at least the following characteristics:
a) Protease activity;
b) Cellulase activity;
c) Xylanase activity;
d) Amylase activity;
e) Activity against antinutritional factors (ANFs), in particular against ß-conglycinine and/or glycinine;
f) Activity against pathogenic microorganisms, in particular against C. perfringens and/or S. suis.

The fermentation broths of the invention, in particular the dried fermentation broths, preferably contain at least five, more preferably at least 6, 7, 8, 9, 10 or 12, metabolites. The metabolites possess preferably a molecular weight of between 200 and 5000 Dalton, more preferably of between 300 and 4000 Dalton.

Further subject matter of the invention are also compositions, in particular feed compositions, containing at least one fermentation broth, in particular at least one dried fermentation broth, according to the invention, wherein the feed composition preferably comprises at least on further feed additive, in particular as disclosed further below.

A further subject matter of the invention is in particular a composition, in particular a feed composition, containing mixtures of different kinds of fermentation broths, in particular different kinds of dried fermentation broths, as mentioned before in the description, wherein the feed composition preferably comprises at least one further feed additive, in particular as disclosed further below.

The fermentation broths of the invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce the pathogen shedding through the feces of such animals. In particular fermentation broths and compositions of the invention might be used to assist in re-establishing a healthy balance of the gut microflora after administration of antibiotics for therapeutic purposes.

A further subject of the invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or of reducing the pathogen shedding through the feces of animals, wherein the fermentation broths of the invention or the compositions of the invention, which comprise such fermentation broths, are administered to animals.

“Increasing the productivity of animals” refers in particular to any of the following: production of more or higher quality eggs, milk or meat or increased production of weaned offspring.

The fermentation broths according to the invention can also be used for improving the quality of water. A further subject of the invention is therefore also a method of controlling and/or improving the quality of water or aqueous solutions, in particular of drinking water and/or rearing water, comprising the step of applying to water a fermentation broth according to the invention.

Further, the fermentation broths according to the invention can also be used for treating plants, in particular for treating microbial diseases of plants. A further subject of the invention is therefore also a method of treating plants, in particular a method of treating and/or preventing microbial diseases of plants, in particular of cultivated plants, comprising the step of applying to the plants at least one fermentation broth of the invention. The application may be carried out in liquid form, such as by spraying, or in solid form, in particular as a powder.

In particular, the fermentation broths of the invention may be administered or fed to an animal in an amount effective to inhibit and/or decrease the growth of pathogenic bacteria in the animal gut. Such pathogenic bacteria include Clostridia, Listeria, Salmonella, Enterococci, Staphylococci, Aeromonas, Streptococci, Campylobacter, Escherichia coli, and Vibrio. Relatedly, the methods of the present invention may be used to decrease the amount of pathogenic bacteria shed in animal feces. The methods of the present invention may also be used to maintain or increase the growth of beneficial bacteria, such as lactic acid bacteria, in the animal gut. By decreasing pathogenic bacteria and/or increasing or maintaining beneficial bacteria, the compositions of the present invention are able to maintain an overall healthy gut microflora.

Thus, a further subject of the invention is a method of inhibiting and/or decreasing the growth of harmful or pathogenic bacteria and/or maintaining and/or increasing the growth of beneficial bacteria in an animal gut, wherein the fermentation broths of the invention are administered to animals and wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gallinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gallinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

In a preferred embodiment of the invention the amount of at least one pathogenic bacterium, in particular the amount of C. perfringens, is reduced by at least 0.5 log, more preferably by at least 1 log, 2 log, or 3 log.

Thus, a further subject of the invention are also fermentation broths of the invention for inhibiting and/or decreasing the growth of pathogenic bacteria and/or for maintaining and/or increasing the growth of beneficial bacteria in an animal gut, wherein the pathogenic bacteria are preferably selected from Clostridia, in particular C. perfringens and C. difficile, Listeria, in particular L. monocytogenes, L. seeligeri and L. welshimeri, Salmonella, in particular S. enterica, S. gallinarum, S. pullorum, S. arizonae, S. typhimurium, S. enteritidis, and S. bongori, Enterococci, in particular E. faecalis, E. faecium and E. cecorum, Staphylococcus, in particular S. aureus, Aeromonas, Streptococci, in particular S. suis and S. gallinaceus, Campylobacter, in particular C. jejuni and C. coli, Escherichia coli, and Vibrio, in particular V. parahemolyticus and V. harveyi, and the beneficial bacteria are preferably selected from lactic acid bacteria, in particular from Lactobacilli, and Bifidobacteria.

The occurrence and/or increased growth of the pathogenic bacteria does or can lead to the outbreak of certain diseases. For example the occurrence and/or increased growth of Clostridium perfringens can lead to the outbreak of gut diseases, in particular to the outbreak of necrotic enteritis in poultry. The occurrence and/or increased growth of Clostridium perfringens can also lead to the outbreak of further diseases like bacterial enteritis, gangrenous dermatitis and colangiohepatitis. Even the mildest form of infection by C. perfringens can already be accompanied by diarrhea, which results in wet litter and by that may lead to secondary diseases like foot pad dermatitis.

A further subject of the invention is therefore also a therapeutic composition comprising at least one fermentation broth of the invention as mentioned before.

A preferred subject in this context is therefore a therapeutic composition for treatment and/or prevention of necrotic enteritis, in particular sub-clinical necrotic enteritis, in animals, preferably poultry, comprising at least one fermentation broth of the invention as mentioned before.

Another preferred subject in this context is therefore a therapeutic composition for treatment and/or prevention of bacterial enteritis, gangrenous dermatitis, colangiohepatitis, clostridiosis, diarrhea and/or foot pad dermatitis, in animals, preferably poultry, comprising at least one fermentation broth as mentioned before.

A further subject of the invention is therefore also the treatment and/or prevention of a disease, in particular of a gut disease, preferably of necrotic enteritis, in particular of sub-clinical necrotic enteritis, in poultry, wherein at least one fermentation broth of the invention is administered to an animal in need thereof.

A further subject of the invention is therefore also the treatment and/or prevention of a disease, preferably a disease of poultry, selected from bacterial enteritis, gangrenous dermatitis, colangiohepatitis, clostridiosis, diarrhea and/or foot pad dermatitis, wherein at least one fermentation broth of the invention is administered to an animal in need thereof.

The fermentation broths of the invention can be administered to animals in feed and/or drinking water over multiple days throughout the animal's life or during particular stages or portions of the animal's life. For example, the strains and/or compositions can be administered only in a starter diet or only in a finisher diet of farm animals.

The compositions of the present invention, in particular the feed, food and pharmaceutical compositions as well as the drinking or rearing water, preferably comprise the fermentation broths of the invention in an amount of from 0.1 wt.-% to 10 wt.-%, more preferably of from 0.2 wt.-% to 5 wt.-%, in particular from 0.3 wt.-% to 3 wt.-%.

The methods of the present invention may be used for all kind of animals, in particular all kind of non-human and non-insect animals, more preferably all kind of vertebrates such as mammals, aquatic animals and birds.

Animals that may benefit from the invention include but are not limited to farm animals, pets, exotic animals, zoo animals, aquatic animals, animals used for sports, recreation or work.

Pets are preferably selected from dogs, cats, domestic birds and domestic exotic animals.

Aquatic animals are preferably selected from finfish and crustaceans which are preferably intended for human nutrition. These include, in particular, carp, tilapia, catfish, tuna, salmon, trout, barramundi, bream, perch, cod, shrimps, lobster, crabs, prawns and crayfish. Preferred types of salmon in this context are the Atlantic salmon, red salmon, masu salmon, king salmon, keta salmon, coho salmon, Danube salmon, Pacific salmon and pink salmon.

Further preferred aquatic animals are farming fish which are subsequently processed to give fish meal or fish oil. In this connection, the fish are preferably herring, pollack, menhaden, anchovies, capelin or cod.

In a further preferred embodiment, the animals are farm animals, which are raised for consumption or as food-producers, such as poultry, swine and ruminants.

The poultry may be selected from productive or domestic poultry, but also from fancy poultry or wild fowl. Preferred productive poultry in this context are chickens, turkeys, ducks and geese. The productive livestock in this context is preferably poultry optimized for producing young stock or poultry optimized for bearing meat. Preferred fancy poultry or wild fowl are peacocks, pheasants, partridges, chukkars, guinea fowl, quails, capercaillies, grouse, pigeons and swans, with quails being especially preferred. Further preferred poultry are ratites, in particular ostriches and emus, as well as parrots.

Ruminants according to the invention are preferably selected from cattle, goat and sheep. In one embodiment, the compositions of this invention may be fed to preruminants to enhance their health and, in particular, to decrease the incidence of diarrhea in these animals. Preruminants are ruminants, including calves, ranging in age from birth to about twelve weeks.

The compositions of the invention may comprise at least one carrier or typical feed additives or combinations thereof.

Suitable carriers are inert formulation additives added to improve recovery, efficacy, or physical properties and/or to aid in packaging and administration. Such carriers may be added individually or in combination. These carriers may be selected from anti-caking agents, anti-oxidation agents, bulking agents, and/or protectants. Examples of useful carriers include polysaccharides (in particular starches, maltodextrins, methylcelluloses, gums, chitosan and/or inulins), protein sources (in particular skim-milk powder and/or sweet-whey powder), peptides, sugars (in particular lactose, trehalose, sucrose and/or dextrose), lipids (in particular lecithin, vegetable oils and/or mineral oils), salts (in particular sodium chloride, sodium carbonate, calcium carbonate, chalk, limestone, magnesium carbonate, sodium phosphate, calcium phosphate, magnesium phosphate and/or sodium citrate), and silicates (in particular clays, in particular beolite clay, amorphous silica, fumed/precipitated silicas, zeolites, Fuller's earth, baylith, clintpolite, montmorillonite, diatomaceous earth, talc, bentonites, and/or silicate salts like aluminium, magnesium and/or calcium silicate).

Suitable carriers for animal feed additives are set forth in the American Feed Control Officials, Inc.'s Official Publication, which publishes annually. See, for example Official Publication of American Feed Control Officials, Sharon Krebs, editor, 2006 edition, ISBN 1-878341-18-9. The carriers can be added after concentrating the fermentation broth and/or during and/or after drying. Preferred carriers according to the invention are selected from calcium carbonate, diatomaceous earth and vegetable oil.

The compositions, in particular feed compositions, of the invention may also comprise probiotics as an additional feed additive, wherein the probiotics are preferably selected from the list of probiotics as mentioned before, i.e. from Bacillus, in particular B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, Enterococcus, in particular E. faecium and E. faecalis, Geobacillus, in particular G. stearothermophilus, Clostridium, in particular C. butyricum, and Streptococcus, in particular S. faecalis, S. faecium, S. gallolyticus, S. salivarius subsp. thermophilus and S. bovis, Lactobacillus, in particular L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius and L. sanfranciscensis, Pediococcus, in particular P. acidilactici, P. dextrinicus and P. pentosaceus, Streptococcus, in particular S. lactis and S. thermophiles, Bifidibacterium, in particular S. adolescentis, B. animalis, B. bifidum, B. breve and B. longum, wherein in a very preferred embodiment of the invention the fermentation broths are from probiotic microorganisms of the genus Bacillus, in particular selected from the following strains and combinations thereof: B. subtilis DSM 32315, B. subtilis DSM 32540, B. subtilis DSM 32592, B. licheniformis DSM 32314, B. pumilus DSM 32539, B. amyloliquefaciens CECT 5940. Further suitable probiotics are selected from Bacillus subtilis PB6 (as described in U.S. Pat. No. 7,247,299 and deposited as ATCC Accession No. PTA-6737), which is sold by Kemin under the trademark CLOSTAT®, Bacillus subtilis C-3102 (as described in U.S. Pat. No. 4,919,936 and deposited as FERM BP-1096 with the Fermentation Research Institute, Agency of Industrial Science and Technology, in Japan), sold by Calpis as CALSPORIN®, Bacillus subtilis DSM 17299, as sold by Chr. Hansen under the trademark GalliPro®, Bacillus licheniformis DSM 17236, as sold by Chr. Hansen under the trademark GalliProTect®, a mixture of Bacillus licheniformis DSMZ 5749 and Bacillus subtilis DSMZ 5750 spores, as sold by Chr. Hansen under the trademark BioPlus®YC, B. subtilis DSM 29784, as sold by Adisseo/Novozymes under the trademark Alterion®, Bacillus subtilis, as sold by Chr. Hansen under the trademark PORCBOOST®, or Bacillus coagulans strains as described in U.S. Pat. No. 6,849,256. Other non-Bacillus probiotics, such as Saccharomyces cerevisiae, Pichia pastoris, Aspergillus niger, Aspergillus oryzae, or Hansenula, may also be used in compositions of the present invention.

Suitable typical animal feed additives which may be also contained in the compositions according to the invention and/or used in the preparation of feed compositions starting from concentrated or dried fermentation broths according to the invention include one or more of the following: proteins, carbohydrates, fats, prebiotics, enzymes, vitamins, immune modulators, milk replacers, minerals, amino acids, coccidiostats, acid-based products and/or medicines, such as antibiotics.

Carbohydrates containing components which may be used according to the invention are for example forage, roughage, wheat meal, sunflower meal or soya meal, and mixtures thereof.

Proteins containing components which may be used according to the invention are for example soya protein, pea protein, wheat gluten or corn gluten, and mixtures thereof.

Fats containing components which may be used according to the invention are in particular oils, of both animal and plant origin, like vegetable oils, for example soya bean oil, rapeseed oil, sunflower seed oil, flaxseed oil or palm oil, fish oil, and mixtures thereof.

Proteins containing components which additionally contain fats which may be used according to the invention are for example fish meal, krill meal, bivalve meal, squid meal or shrimp shells, as well as combinations thereof.

Prebiotics which may be used according to the invention are preferably oligosaccharides, in particular selected from galactooligosaccharides, silayloligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides, xylooligosaccharides, dextrans, pectins, polygalacturonan, rhamnogalacturonan, mannan, hemicellulose, arabinogalactan, arabinan, arabinoxylan, resistant starch, mehbiose, chitosan, agarose, inulin, tagatose, polydextrose, and alginate.

Enzymes which may be used in feed compositions according to the invention and which may aid in the digestion of feed, are preferably selected from phytases (EC 3.1.3.8 or 3.1.3.26), xylanases (EC 3.2.1.8), galactanases (EC 3.2.1.89), galactosidases, in particular alpha-galactosidases (EC 3.2.1.22), proteases (EC 3.4), phospholipases, in particular phospholipases A1 (EC 3.1.1.32), A2 (EC 3.1.1.4), C (EC 3.1.4.3), and D (EC 3.1.4.4), lysophospholipases (EC 3.1.1.5), amylases, in particular alpha-amylases (EC 3.2.1.1); lysozymes (EC 3.2.1.17), glucanases, in particular beta-glucanases (EC 3.2.1.4 or EC 3.2.1.6), glucoamylases, cellulases, pectinases, or any mixture thereof.

Examples of commercially available phytases include Bio-Feed™ Phytase (Novozymes), Ronozyme® P and HiPhos™ (DSM Nutritional Products), Natuphos™ (BASF), Finase® and Quantum@ Blue (AB Enzymes), the Phyzyme® XP (Verenium/DuPont) and Axtra® PHY (DuPont). Other preferred phytases include those described in e.g. WO 98/28408, WO 00/43503, and WO 03/066847.

Examples of commercially available xylanases include Ronozyme® WX and G2 (DSM Nutritional Products), Econase® XT and Barley (AB Vista), Xylathin® (Verenium) and Axtra® XB (Xylanase/beta-glucanase, DuPont). Examples of commercially available proteases include Ronozyme® ProAct (DSM Nutritional Products).

Vitamins which may be used according to the invention are for example vitamin A, vitamin D3, vitamin E, vitamin K, e.g., vitamin K3, vitamin B12, biotin, choline, vitamin B1, vitamin B2, vitamin B6, niacin, folic acid and panthothenate, e.g., Ca-D-panthothenate, or combinations thereof.

Immune modulators which may be used are for example antibodies, cytokines, spray-dried plasma, interleukins, or interferons, or combinations thereof.

Minerals which may be used according to the invention are for example boron, cobalt, chloride, chromium, copper, fluoride, iodine, iron, manganese, molybdenum, selenium, zinc, calcium, magnesium, potassium, or sodium, or combinations thereof.

Amino acids which may be used according to the invention are for example lysine, alanine, threonine, methionine, valine or tryptophan, or combinations thereof.

Thus, a further embodiment of the invention is a method of preparing an animal feed composition comprising mixing at least one fermentation broth, in particular a dried fermentation broth, or a mixture of fermentation broths according to the invention, in particular in an amount effective to enhance animal health, with feed additives, such as proteins, lipids and/or carbohydrates, and optionally further beneficial substances, preferably as mentioned before, to provide a feeding product. This method may comprise for example also a pelleting step.

Standard pelleting processes known to those of skill in the art may be used, including extrusion processing of dry or semi-moist feeds. Preferred pelleting temperatures are between about 65° C. and about 120° C.

In a particularly preferred embodiment of the invention, in preparing a feed composition of the invention, the fermentation broth(s) according to the invention is/are added in a subsequent step onto an already prepared feed product, in particular onto a feed pellet, wherein adding the fermentation broth(s) to the already prepared feed product, in particular feed pellet, is preferably carried out by spraying or vacuum coating. This method has the particular advantage that thermal degradation of the enzymes as contained in the fermentation broth(s) can be completely avoided.

In addition other sensitive materials like oils and/or enzymes might be added to the feed product, in particular to a feed pellet, by spraying or vacuum coating.

The fermentation broths of the present invention can be obtained by culturing the strains of the invention according to methods well known in the art, including by using the media, conditions and methods as described for example in U.S. Pat. No. 6,060,051, EP0287699, US2014/0010792 or FAO Report 179 (2016): Probiotics in Animal Nutrition. Conventional large-scale microbial culture processes include submerged fermentation, solid state fermentation, or liquid surface culture. Towards the end of fermentation, as nutrients are depleted, the cells of the Bacillus strains begin the transition from growth phase to sporulation phase, such that the final product of fermentation is largely spores, metabolites and residual fermentation medium. Sporulation is part of the natural life cycle of these strains and is generally initiated by the cell in response to nutrient limitation. Fermentation is configured to obtain high levels of colony forming units of the probiotic cells and to promote sporulation.

Preferably according to the invention always an effective amount of the fermentation broths of the invention is used in the embodiments of the invention. The term “effective amount” refers to an amount which effects at least one beneficial effect to an animal and/or to the environment, in particular with respect to the features as already mentioned before, in comparison to an animal that has not been administered the fermentation broth of the invention, but besides that has been administered the same diet (including feed and other compounds).

In case of therapeutic applications preferably a therapeutic amount of the fermentation broths of the invention is used. The term “therapeutic amount” refers to an amount sufficient to ameliorate, reverse or prevent a disease state in an animal. Optimal dosage levels for various animals can easily be determined by those skilled in the art, by evaluating, among other things, the composition's ability to (i) inhibit or reduce pathogenic bacteria in the gut at various doses, (ii) increase or maintain levels of beneficial bacteria and/or (iii) enhance animal health at various doses.

### EXPLANATION OF THE FIGURES

FIG. 1 shows the cellulase activity of a) vegetative cells of the B. subtilis strain DSM 32540; b) vegetative cells of B. amyloliquefaciens strain CECT 5940; c) sterile filtrated non-dried supernatant of B. amyloliquefaciens CECT 5940 fermentation; d) sterile filtrated non-dried supernatant of B. amyloliquefaciens CECT 5940 fermentation. Cellulase activity leads to a clearance on the agar plate around the zone of cellulose hydrolysis.

FIG. 2 shows the cellulase activity of a) vegetative cells of B. subtilis strain DSM 32540; b) sterile filtrated supernatant of B. subtilis DSM 32540 fermentation; c) sterile filtrated non-dried supernatant of a B. subtilis DSM 32540 fermentation; d) non-sterile filtrated non-dried supernatant of B. subtilis DSM 32540 fermentation; e) non-sterile filtrated supernatant of B. subtilis DSM 32540 fermentation. Cellulase activity leads to a clearance on the agar plate around the zone of cellulose hydrolysis.

FIG. 3 shows cellulase activity of a)-d) sterile filtrated (freeze-dried and dissolved) supernatant of B. amyloliquefaciens CECT 5940 fermentation; e) vegetative cells of B. amyloliquefaciens CECT 5940. Cellulase activity leads to a clearance on the agar plate around the zone of cellulose hydrolysis.

FIG. 4 shows the xylanase activity of a) vegetative cells of B. amyloliquefaciens CECT 5940; b) vegetative cells of B. subtilis DSM 32540; c) sterile filtrated non-dried supernatant of B. amyloliquefaciens CECT 5940 fermentation; d) sterile filtrated non-dried supernatant of a B. amyloliquefaciens CECT 5940 fermentation. Xylanase activity leads to a clearance on the agar plate around the zone of xylan hydrolysis.

FIG. 5 shows the amylase activity of a)-d) sterile filtrated (freeze-dried and dissolved) supernatant of B. amyloliquefaciens CECT 5940 fermentation; e) vegetative cells of B. amyloliquefaciens CECT 5940 strain. Amylase activity leads to a clearance on the agar plate around the zone of starch hydrolysis.

FIG. 6 shows the protease activity of a) vegetative cells of a B. subtilis DSM 32540; b) vegetative cells of B. amyloliquefaciens CECT 5940; c) sterile filtrated non-dried supernatant of B. amyloliquefaciens CECT 5940 fermentation; d) sterile filtrated non-dried supernatant of B. amyloliquefaciens CECT 5940 fermentation. Protease activity leads to a clearance on the agar plate around the zone of substrate hydrolysis.

FIG. 7 shows the protease activity of a)-d) sterile filtrated (freeze-dried and dissolved) supernatant of B. amyloliquefaciens CECT 5940 fermentation; e) vegetative cells of a B. amyloliquefaciens CECT 5940. Protease activity leads to a clearance on the agar plate around the zone of substrate hydrolysis.

FIG. 8 shows the SDS-PAGE pattern of soy protein extracts treated for 24 h with sterile filtrated non-dried supernatants of fermentation of B. subtilis DSM 32540.

FIG. 9 shows the SDS-PAGE pattern of soy protein extracts treated for 24 h with sterile filtrated non-dried supernatants of fermentation of B. amyloliquefaciens CECT 5940. Indicated protein bands were identified via nLC/MS analyses as: 1-Beta-conglycinin, alpha′ chain of Glycine max; 2-Beta-conglycinin alpha subunit of Glycine max; 3-Beta-conglycinin alpha subunit of Glycine max; 4-Glycinin of Glycine max; 5-Beta-conglycinin alpha prime subunit of Glycine max; 6-Beta-conglycinin alpha prime subunit of Glycine max; 7-Beta-conglycinin alpha subunit of Glycine max.

### WORKING EXAMPLES

### Example 1. Qualitative and Quantitative Assessment of Digestive Enzymatic Activities

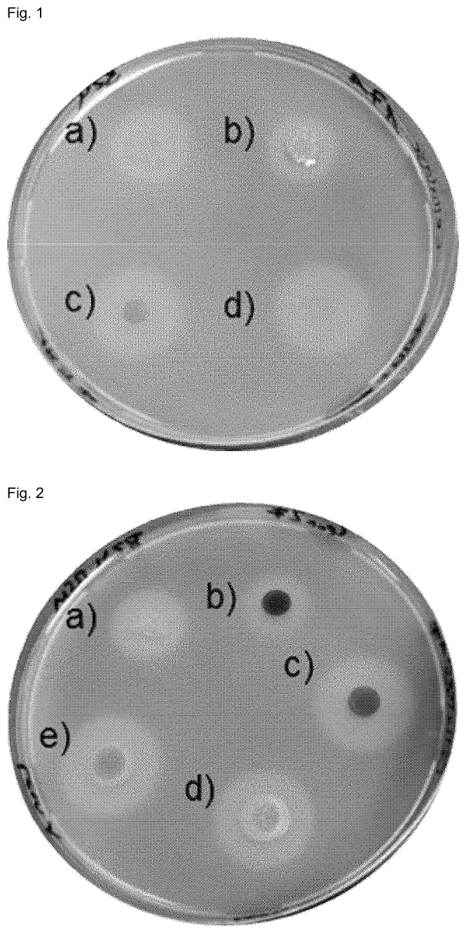

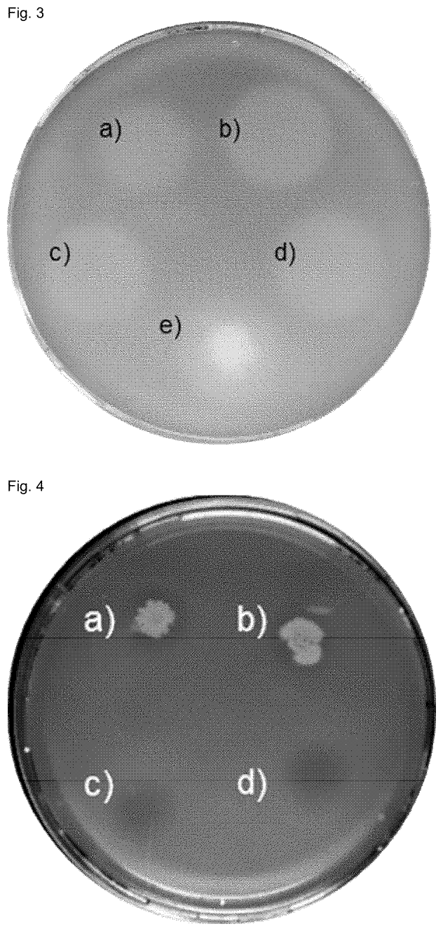

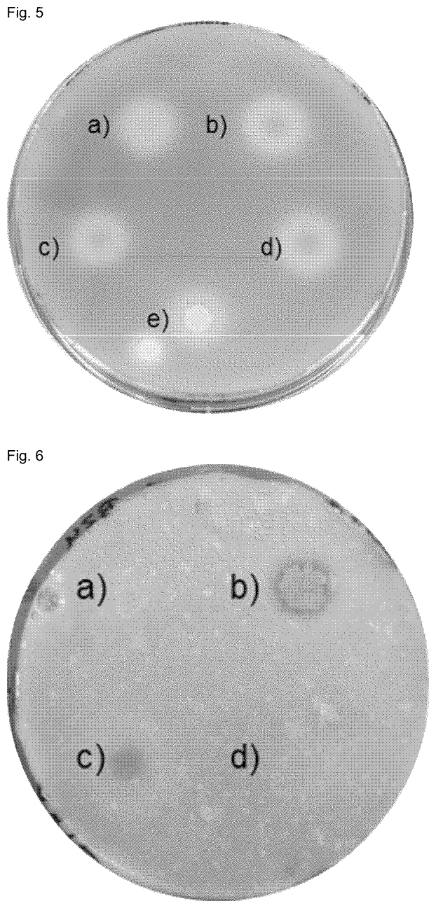

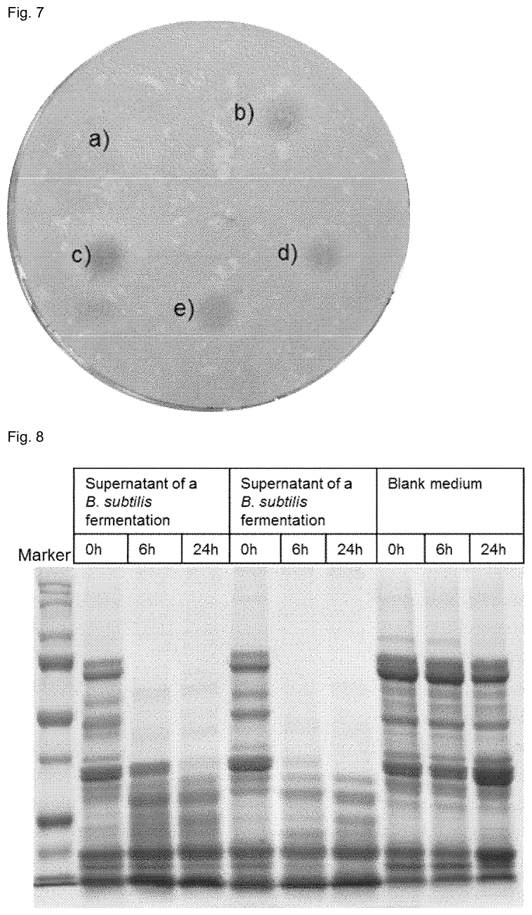

Supernatants of a fermentation in a standard medium of a probiotic B. subtilis DSM 32540 and B. amyloliquefaciens strain CECT 5940 were evaluated for digestive enzyme activities, in particular aerobic cellulytic (FIG. 1-3), xylanolytic (FIG. 4), amylase (FIG. 5) and proteolytic activity (FIG. 6-7).

For the evaluation of cellulase activity, 3 μl of sterile filtrated non-dried and/or freeze-dried and dissolved supernatant of probiotic Bacillus fermentations were spotted onto LB agar containing 5 g/I Sigmacell Cellulose. To screen for protease activity, 3 μl sterile filtrated non-dried and/or freeze-dried and dissolved supernatant of probiotic Bacillus strain fermentations were spotted onto LB agar containing 10% skim milk. Xylanase activity was analyzed in a similar way on LB agar containing 0.5% xylan; amylase activity was analyzed on LB agar containing 10 g/I soluble starch.

As positive control, enzymatic activities of vegetative cells of the respective probiotic strains were analyzed. Therefore, 3 μl of a liquid culture were spotted directly onto the respective agar plates, which were incubated at 37° C. under aerobic conditions. The read out parameter was the appearance of hydrolysis zones resulting from the enzymatic activities. The plates used in the cellulase and amylase assay were stained with Lugol's iodine solution (FIG. 1-3; 5).

Analyses of digestive enzyme activities in a qualitative way have shown that respective activities can be found in sterile filtrated supernatants as well as in freeze-dried and dissolved sterile filtrated supernatants.

In addition, proteolytic activity of sterile filtrated non-dried supernatants of Bacillus strain fermentations was assessed in a quantitative way. 10 μL sterile filtrated supernatant were added to 20 μL 0.5% Fluorescein Isothiocyanate Casein (FITC; C3777, Sigma-Aldrich) solution with 20 μL buffer consisting of 20 mM sodium phospate (dibasic, anhydrous) with 150 mM sodium chloride (all components from Sigma-Aldrich), then incubated for 1 h at 37° C. After addition of 150 μL of 10% (v/v) trichlor acetic acid (Sigma-Aldrich) and another 30 min incubation at 37° C., samples were centrifuged at 19,000 rpm for 15 min, then 2 μL of supernatant transferred to 200 μL 500 mM TRIS HCl Solution (Trizma BaseTRIS, Sigma-Aldrich). Fluorescence of soluble peptides due to proteolytic release were determined (TECAN GENios Microplate Reader, Tecan Group Ltd., Mannedorf, Switzerland) at excitation 494 nm, emission 518 nm. Analysis was performed in two independent runs, then averaged as milliunits per microliter solution. Results can be found in Table 1.

TABLE 1
Protease activity of sterile filtrated supernatants of B. subtilis DSM 32540
and a B. amyloliquefaciens CECT 5940 fermentation.

| | Protease activity |
|---|---|
| Sterile filtrated supernatant of | (mU/mL) |
| a fermentation of a B. subtilis strain | 107.56 ± 1.98 |
| a fermentation of a B. amyloliquefaciens strain | 1551.97 ± 118.06 |

In direct comparison, the supernatant of the fermentation of the B. amyloliquefaciens CECT 5940 has a more than 10 fold increased protease activity than the supernatant of the B. subtilis DSM 32540 fermentation.

### Example 2. Pathogen Inhibition by Supernatants of Fermentations of Probiotic Bacillus Strains

Pathogen inhibition by the supernatant via secondary metabolites produced by probiotic Bacillus strains during fermentation was assessed using well diffusion antagonism tests (Parente et al. 1995).

A well diffusion antagonism test with different pathogens, Clostridium perfringens type strain ATCC 13124 from Teo and Tan (2005) and Streptococcus suis ATCC 43765 was performed (assay performed with freeze-dried dissolved sterile filtrated supernatant). Strain ATCC 13124 is known to be a alpha-toxigenic Type A strain serving as a type strain for Clostridia.

S. suis is an important pathogen in pigs and one of the most important causes of bacterial mortality in piglets after weaning causing septicemia, meningitis and many other infections (Goyette-Desjardins et al. 2014). ATCC 43765 belongs to Serological group: R; serovar 2 and was isolated from pigs.

The pathogenic strains were grown under suitable conditions as liquid culture to an optical density of 600 nm of at least 1, then 130 μl were spread with a sterile spatula on the surface of agar plates. For all pathogens TSBYE agar plates are used. 9 mm diameter wells were cut into the dried plates.

The 1st well was used as non-inoculated media control without culture, the other wells were inoculated with 100 μL sterile filtrated supernatant (with or without heat treatment) of fermentations of probiotic Bacillus strains. After 24 h incubation under suitable conditions at 37° C., the zone of clearance in mm was determined measuring from the edge of the cut well to the border of the cleared lawn. Each colony was measured twice (horizontally, vertically), then averaged. The results can be found in the following tables 2 and 3.

TABLE 2
Comparison of heat treated and non-heat treated sterile
filtrated non-dried supernatant of B. amyloliquefaciens
CECT 5940 fermentation in inhibitory capacity on a
pathogenic Clostridium perfringens strain in a well
diffusion antagonism assays on TSBYE medium, values
in mm clearance of pathogen.

| | Pathogen |
|---|---|
| | C. perfringens ATCC |
| Supernatant | 13124 (mm) |
| 2 min 85° C. heat treated sterile | 29.15 |
| filtrated supernatant of a | |
| fermentation of the B. amyloliquefaciens | |
| strain CECT 5940 | |
| sterile filtrated supernatant of a | 28.38 |
| fermentation of a B. amyloliquefaciens | |
| strain CECT 5940 | |

The data show that the non-dried supernatant of a B. amyloliquefaciens CECT 5940 fermentation—also heat treated-inhibits the growth of C. perfringens very effectively.

TABLE 3
Comparison of heat treated and non-heat treated sterile filtrated
supernatant of B. subtilis DSM 32540 fermentation
in inhibitory capacity on a pathogenic S. suis strain in
a well diffusion antagonism assays on TSBYE medium,
values in mm clearance of pathogen.

| | Pathogen |
|---|---|
| | S. suis ATCC |
| Supernatant | 43765 (mm) |
| 2 min 85° C. heat treated sterile filtrated | 22.64 |
| supernatant (freeze dried and dissolved in water | |
| again) of a fermentation of B. subtilis strain DSM | |
| 32540 | |
| sterile filtrated supernatant (freeze dried and | 22.60 |
| dissolved in water again) of a fermentation of B. | |
| subtilis strain DSM 32540 | |

The data show that the supernatant of a B. subtilis DSM 32540 fermentation—also heat treated-inhibits the growth of S. suis ATCC 43765 very effectively. Additionally, also after freeze-drying an inhibitory effect could still be observed.
Teo, A. Y.-L. and Tan, H.-M. (2005). Inhibition of Clostridium perfringens by a novel strain of Bacillus subtilis from the gastrointestinal tracts of healthy chickens. Appl. Environm. Microbiol., 71:4185-90.
Parente, E., Brienza, C., Moles, M., & Ricciardi, A. (1995). A comparison of methods for the measurement of bacteriocin activity. Journal of microbiological methods, 22(1), 95-108.
Goyette-Desjardins, G., Auger, J. P., Xu, J., Segura, M. and Gottschalk, M. (2014). Streptococcus suis, an important pig pathogen and emerging zoonotic agent—an update on the worldwide distribution based on serotyping and sequence typing. Emerg Microbes Infect. 2014 June; 3(6):e45.

### Example 3. Assessment of Hydrolytic Activity of Sterile Filtrated Supernatants of Probiotic Bacillus Strain Fermentations on Antinutritional Factors of Soybean Meal

Proteins were extracted from defatted soybean meal using a method adapted from Iwabuchi and Yamauchi (1987). Defatted soybean meal was extracted using 100 ml 0.03 M Tris-HCl (pH 8) containing 10 mM R-mercaptoethanol with agitation for 1 h at room temperature. Samples were centrifuged, the supernatant sterile filtrated and samples stored at −20° C.

Sterile filtrated non-dried supernatants of probiotic B. subtilis DSM 32540 and B. amyloliquefaciens CECT 5940 fermentations were incubated in a 2:1 ratio with soy protein extracts at 37° C. Samples were taken at 0 hrs, 6 hrs and 24 hrs, centrifuged and the supernatant stored at −20° C. for further analyses. Control samples with the addition of non-spend medium were analyzed in parallel.

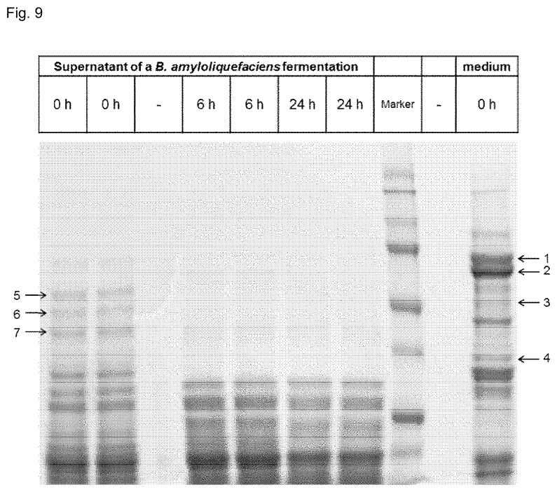

Protein concentrations were determined using the Bio-Rad Protein Assay Kit (Bio-Rad, USA). Protein hydrolysis was monitored using SDS-Page. Protein concentrations were adjusted and proteins denatured by 5 min 95° C. heat treatment before loading onto the gel. 20 μg of extracted proteins were loaded into each well of a 10% Mini Protean TGX Precast SDS Gel. The Precision Plus Protein™ Dual Color Standards protein ladder was used as marker (10-250 kDa). Proteins were separated at 40 mA for an hour. Gels were stained with a Coomassie Brilliant Blue G250 and Coomassie Brilliant Blue R250 solution and destained with an acetic acid solution. Degradation of soy proteins could be observed by the disappearance of proteins bands over time (FIGS. 8 and 9).

Proteolytic degradation of soy protein extracts was undetectable in control samples containing medium and soy protein extract only. Hydrolysis of prominent proteins could be detected after 6 and 24 h incubation of soy protein extract with a sterile filtrated supernatant of a B. subtilis DSM 32540 fermentation and B. amyloliquefaciens CECT 5940 fermentation, respectively. An increase in of smaller peptides (<25 kDa) accompanied the decrease of multiple bigger protein bands 25-75 kDa). Protein bands that were degraded over time and specific bands from the control sample of the soy extract incubated with medium only were analyzed by nano-LC/MS and identified via high-resolution mass spectrometry to be ß-conglycinine and glycinine of Glycine max. Thus, the antigenic proteins R-conglycinin and glycinin of soy were degraded during the incubation with non-dried sterile filtrated supernatant of a Bacillus fermentations.
Iwabuchi, S. and Yamauchi, F. (1987): Determination of glycinin and R-conglycinin in soybean proteins by immunological methods. J. Agric. Food Chem. 35, 200-205.

1-19. (canceled)

20. A method of producing a dried fermentation broth, comprising the following steps:
a) cultivating microorganisms in a fermentation medium to obtain a fermentation broth containing the microorganisms;
b) separating at least 20% of the microorganisms from the fermentation broth; and
c) drying the fermentation broth of step b) to obtain the dried fermentation broth.

21. The method of claim 20, wherein drying of the fermentation broth is carried out by freeze-drying, spray drying, vacuum drying, fluidized bed drying or spray granulation.

22. The method of claim 20, wherein the microorganisms are selected from the group consisting of: B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis and B. vallismortis, E. faecium, E. faecalis, G. stearothermophilus, C. butyricum, S. faecalis, S. faecium, S. gallolyticus, S. salivarius subsp. thermophiles, S. bovis, L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius, L. sanfranciscensis, P. acidilactici, P. dextrinicus, P. pentosaceus, S. lactis, S. thermophiles, S. adolescentis, B. animalis, B. bifidum, B. breve and B. longum.

23. Dried fermentation broth of microorganisms, obtainable by cultivating at least one microorganism in a fermentation medium, removing at least 20%, of the microorganisms from the fermentation broth, and subsequently drying the fermentation broth thus obtained, wherein the dried fermentation broth contains microorganisms in an amount of less than 1 wt.

24. The dried fermentation broth of claim 23, further comprising at least one substance selected from the group consisting of: anti-caking agents, anti-oxidation agents, bulking agents, protectants, polyethylene glycol, amino acids, protein sources, peptides, sugars, polyols, yeast extract, malt extract, soybean flour, lipids, salts, and silicates.

25. The dried fermentation broth of claim 23, wherein the broth is from a fermentation of Bacillus, Enterococcus, Geobacillus, Clostridium, and Streptococcus, Lactobacillus, B. animalis, B. bifidum, B. breve and B. longum, or from mixtures of at least two of such fermentation broths.

26. The dried fermentation broth of claim 25, wherein the broth is from fermentation of Bacillus amyloliquefaciens.

27. The died fermentation broth of claim 23, comprising at least one of the following characteristics:
a) Protease activity;
b) Cellulase activity;
c) Xylanase activity;
d) Amylase activity;
e) Phytase activity;
f) Catalase activity;
g) Superoxide dismutase activity;
h) Lactonase activity;
i) Activity against antinutritional factors (ANFs);
j) Activity against mycotoxins;
k) Activity against pathogenic microorganisms, in particular against C. perfringens and/or S. suis;
l) Quorum quenching activity;
m) Prebiotic activity with respect to beneficial microorganisms.

28. The dried fermentation broth of claim 23, further comprising a feed additive selected from the group consisting of: probiotics and mixtures of probiotics, carbohydrates, fats, prebiotics, enzymes, vitamins, immune modulators, milk replacers, minerals, amino acids, coccidiostats, acid-based products and/or medicines, antibiotics, and mixtures thereof.

29. A method of improving the health or nutrition of animals, plants or the quality of aqueous solutions, comprising administering the dried fermentation broth of claim 4 or a composition comprising the dried fermentation.

30. The method of claim 29, wherein said method comprises feeding animals the dried fermentation broth or the composition comprising the dried fermentation broth.

31. The method of claim 29, wherein said method comprises enhancing the health of animals, and/or improving the general physical condition of animals, and/or improving the feed conversion rate of animals, and/or decreasing the mortality rate of animals, and/or increasing the survival rates of animals, and/or improving the weight gain of animals, and/or increasing the disease resistance of animals, and/or increasing the immune response of animals, and/or establishing or maintaining a healthy gut microflora in animals, and/or reducing pathogen shedding through the feces of animals, and wherein the method comprises administering the dried fermentation broth or a composition comprising the dried fermentation to the animals.

32. The method of claim 29, wherein said method comprises controlling and/or improving the quality of water or an aqueous solution by applying the dried fermentation broth or a composition comprising the dried fermentation broth to the water or aqueous solution.

33. The method of claim 29, wherein said method comprises treating and/or preventing a microbial disease of a cultivated plant by applying the dried fermentation broth or a composition comprising the dried fermentation to the plant.

34. The method of claim 29, wherein said method comprises improving the properties of an animal feed or feed additive, by adding the dried fermentation broth or a composition comprising the dried fermentation to the animal feed or feed additive.

35. The method of claim 34, wherein the properties which are improved are selected from: reducing the amount of antinutritional factors (ANFs), and/or degrading mycotoxins; and/or increasing the usability of proteins by the animals; and/or preserving the feed or feed additive by lowering the pH and/or lowering the amount of contaminating microorganisms in the feed or feed additive.

36. The method of claim 34, wherein the feed additive is selected from corn, soy, barley, rice, oats, sorghum, soybean meal, rapeseed meal and cotton meal.

37. The method of claim 34, wherein the animal feed or feed additive and the fermentation broth are mixed in a ratio of 2:1 to 1:20.

38. The method of claim 34, wherein the animal feed or feed additive and the fermentation broth are incubated for at least 1 hour.

39. The method of claim 34, wherein the fermentation broth is selected from fermentation broths of: B. subtilis, B. licheniformis, B. amyloliquefaciens, B. atrophaeus, B. clausii, B. coagulans, B. flexus, B. fusiformis, B. lentus, B. megaterium, B. mesentricus, B. mojavensis, B. polymixa, B. pumilus, B. smithii, B. toyonensis, B. vallismortis, E. faecium, E. faecalis, G. stearothermophilus, C. butyricum, S. faecalis, S. faecium, S. gallolyticus, S. salivarius subsp. thermophilus and S. bovis; L. acidophilus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paracasei, L. paraplantarum, L. pentosus, L. plantarum, L. pontis, L. reuteri, L. rhamnosus, L. sakei, L. salivarius, L. sanfranciscensis, P. acidilactici, P. dextrinicus, P. pentosaceus, S. adolescentis, B. animalis, B. bifidum, B. breve, B. longum, and mixtures of such fermentation broths.
